(19) United States
(12) Patent Application Publication
(10) Pub. No.: US 2024/0252345 A9
(43) Pub. Date: Aug. 1, 2024

(54) MALE FLUID COLLECTION ASSEMBLIES AND SYSTEMS, METHODS OF USING, AND METHODS OF MANUFACTURING THE SAME

(71) Applicant: PUREWICK CORPORATION, Covington, GA (US)

(72) Inventors: Gregory Mann, Covington, GA (US); Anthony Esposito, Oxford, GA (US); Kathleen Davis, Atlanta, GA (US); Samuel Edmund Whittome, Cambridge (GB); Emily Lucy Triggs, Cambridge (GB); Merissa Lim Sarrias, Hauxton (GB); Jack Warren, North Hertfordshire (GB); Martyn Mitchell, Hertfordshire (GB); Barry Dobson, Manchester (GB); Karen X.Z. Yu, Cambridge (GB); Robert Hine, Convington, GA (US)

(21) Appl. No.: 18/003,029

(22) PCT Filed: Jun. 30, 2021
(86) PCT No.: PCT/US2021/039866

§ 371 Date: Dec. 22, 2022

Related U.S. Application Data
(60) Provisional application No. 63/047,374, filed on Jul. 2, 2020.
(60) Provisional application No. 63/067,542, filed on Aug. 19, 2020.

(51) Int. Cl. A61F 5/453; A61F 5/44; A61F 5/443

(52) U.S. Cl. CPC A61F 5/453; A61F 5/4404; A61F 5/443

(57) ABSTRACT

An example fluid collection assembly (100) includes a sheath (102) and a base (104). The sheath includes a fluid impermeable barrier (106) formed from at least a first panel (108) and a second panel (110). The first panel and the second panel at least partially define a chamber (112) therebetween. The fluid impermeable barrier also defines a fluid outlet (118) at a distal end of the sheath and an opening (114) at a proximal end of the sheath. The sheath also includes at least one porous material (122) disposed in the chamber. The base is permanently secured or configured to be permanently secured to the sheath. The base defines an aperture (124) that is aligned with the opening when the base is permanently attached to the sheath. The base is configured to be secured to a region about a penis of an individual with the aperture positioned over the penis.

### CROSS-SECTION TO RELATED APPLICATIONS

This application claims priority to U.S. Provisional Patent Application No. 63/047,374 filed on Jul. 2, 2020 and U.S. Provisional Patent Application No. 63/067,542 filed on Aug. 19, 2020, the disclosure of each of which is incorporated herein, in its entirety, by this reference.

### BACKGROUND

In various circumstances, a person or animal may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

Urinary catheters, such as a Foley catheter, can be used to address some of these circumstances, such as incontinence. Unfortunately, however, urinary catheters can be uncomfortable, painful, and can lead to complications, such as infections. Additionally, bed pans, which are receptacles used for the toileting of bedridden patients, such as those in a health care facility, are sometimes used. Bed pans, however, can be prone to discomfort, spills, and other hygiene issues.

Males who suffer the most severe consequences of urinary incontinence, such as discomfort, rashes, and sores are typically elderly and often bedbound. They also require continuous assistance to maintain hygiene. Characteristics often found in these patients: they typically lay on their back, the size of the penis often decreases with age, skin rolls containing fat tissue cause the penis to recede, often pointing upward while in a laying position, patients have difficulty reaching the penis and manipulating devices. A urine capture device should be designed with reference to these characteristics.

Available solutions are typically for use while standing up (such as cups and funnels), with a urine discharge port opposite to the distal end of the penis. Other designs such as condom-style catheters are difficult for patients to manipulate, too often they are dimensionally incompatible; and they do not stay on reliably.

Thus, there is a need for a device capable of collecting urine from a person or animal, particularly a male, comfortably and with minimal contamination of the user and/or the surrounding area.

### SUMMARY

Embodiments disclosed herein are directed to male fluid collection assemblies, systems including the same, methods of manufacturing the same, and methods of using the same. In an embodiment, a fluid collection assembly is disclosed. The fluid collection assembly includes a sheath. The sheath includes at least one fluid impermeable barrier including a first panel and a second panel. The at least one fluid impermeable barrier defines a chamber at least partially defined by the first panel and the second panel. The at least one fluid impermeable barrier defines an outlet at a distal end of the sheath and an opening at a proximal end of the sheath. The sheath also includes at least one porous material disposed in the chamber. The fluid collection assembly also includes a base permanently secured to or configured to be permanently secured to the proximal end of the sheath. The base is configured to be attached to skin surrounding a penis. The base defines an aperture that corresponds to the opening of the sheath.

In an embodiment, a system is disclosed. The system includes a fluid collection assembly. The fluid collection assembly includes a sheath. The sheath includes at least one fluid impermeable barrier including a first panel and a second panel. The at least one fluid impermeable barrier defines a chamber at least partially defined by the first panel and the second panel. The at least one fluid impermeable barrier defines an outlet at a distal end of the sheath and an opening at a proximal end of the sheath. The sheath also includes at least one porous material disposed in the chamber. The fluid collection assembly also includes a base permanently secured to or configured to be permanently secured to the proximal end of the sheath. The base is configured to be attached to skin surrounding a penis. The base defines an aperture that corresponds to the opening of the sheath. The system also includes a vacuum source configured to apply a suction force, a fluid storage container, and at least one conduit connected to the outlet and in fluid communication with the vacuum source and the fluid storage container.

In an embodiment, a method of manufacturing a fluid collection assembly is disclosed. The method includes attaching a first panel and a second panel of a fluid impermeable barrier together along at least a portion of edges thereof to form a sheath in which the first and second panels at least partially defining a chamber therebetween. The fluid impermeable barrier defines an opening and an outlet. The method also includes disposing at least one porous material in the chamber.

In an embodiment, a method of using a system to collect bodily fluids from an individual is disclosed. The method includes attaching a base to skin surrounding a penis. The base permanently secured or configured to be permanently secured to a sheath. The sheath includes at least one fluid impermeable barrier including a first panel and a second panel. The at least one fluid impermeable barrier defines a chamber at least partially defined by the first panel and the second panel. The at least one fluid impermeable barrier defines a fluid outlet at a proximal end of the sheath and an opening at a distal end of the sheath. The sheath also includes at least one porous material disposed in the chamber.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIGS. 1A and 1B are top and bottom isometric views, respectively, of a fluid collection assembly, according to an embodiment.

FIGS. 1C and 1D are cross-sectional schematics of the fluid collection assembly taken along planes C-C and D-D, respectively, according to any embodiment.

FIG. 1E is a top plan view of the second panel of the fluid collection assembly, according to an embodiment.

FIG. 1F is a cross-sectional schematic of a porous material, according to an embodiment.

FIG. 1G is a cross-sectional schematic of the base 104, according to an embodiment.

FIG. 2A is a cross-sectional schematic of a fluid collection assembly, according to an embodiment.

FIG. 2B is a top plan view of the second panel, according to an embodiment.

FIG. 3 is a cross-sectional schematic of a fluid collection assembly, according to an embodiment.

FIG. 4A is a cross-sectional schematic of a fluid collection assembly, according to an embodiment.

FIG. 4B is a cross-sectional schematic of the base shown in FIG. 4A, according to an embodiment.

FIG. 5 is a cross-sectional view of a fluid collection assembly that includes a first panel and a second panel integrally formed together, according to an embodiment.

FIGS. 6A and 6B are cross-sectional schematics illustrating how substantially similar fluid collection assemblies may be used with a buried and non-buried penis, according to an embodiment.

FIG. 7 is a block diagram of a system for fluid collection, according to an embodiment.

FIG. 8 is a flow diagram of a method to collect fluid, according to an embodiment.

FIG. 9 is a flow diagram of a method to manufacture a fluid collection assembly, according to an embodiment.

### DETAILED DESCRIPTION

Embodiments disclosed herein are directed to male fluid collection assemblies, systems including the same, methods of manufacturing the same, and methods of using the same. An example fluid collection assembly includes a sheath and a base. The sheath includes a fluid impermeable barrier formed from at least a first panel and a second panel connected together. The first panel and the second panel at least partially define a chamber therebetween. The fluid impermeable barrier also defines a fluid outlet at a distal end of the sheath and an opening at a proximal end of the sheath. The sheath also includes at least one porous material disposed in the chamber. The base may be permanently secured to the sheath or the base may be configured to be permanently secured to the sheath at some time period in the future. The base defines an aperture that is aligned with the opening when the base is permanently attached to the sheath. The base is configured to be secured to a region about a penis of an individual with the aperture positioned over the penis. As such, the chamber of the sheath may be configured to receive one or more bodily fluids (e.g., urine, semen, sweat, etc.) from penis.

An example method of using the fluid collection assembly includes securing the base to the region about the penis of an individual. The base is positioned on the individual such that the penis extends through (e.g., the penis is not buried) or is adjacent to (e.g., the penis is buried) the aperture defined by the base. If sheath is not already attached to the base, the sheath may also be permanently attached to the base. For example, the sheath may be permanently attached to the base before, during, or after securing the base to the region about the penis. After the base is secured to the region about the penis and the sheath is attached to the base, the individual may discharge bodily fluids from the penis. The bodily fluids enter the chamber of the sheath. The porous material may receive at least some of the bodily fluids that enter the chamber and direct the bodily fluids towards the outlet. The method may include removing the bodily fluids from the chamber through the outlet, for instance, when a suction force is applied to the outlet via a vacuum source that is in fluid communication with the chamber.

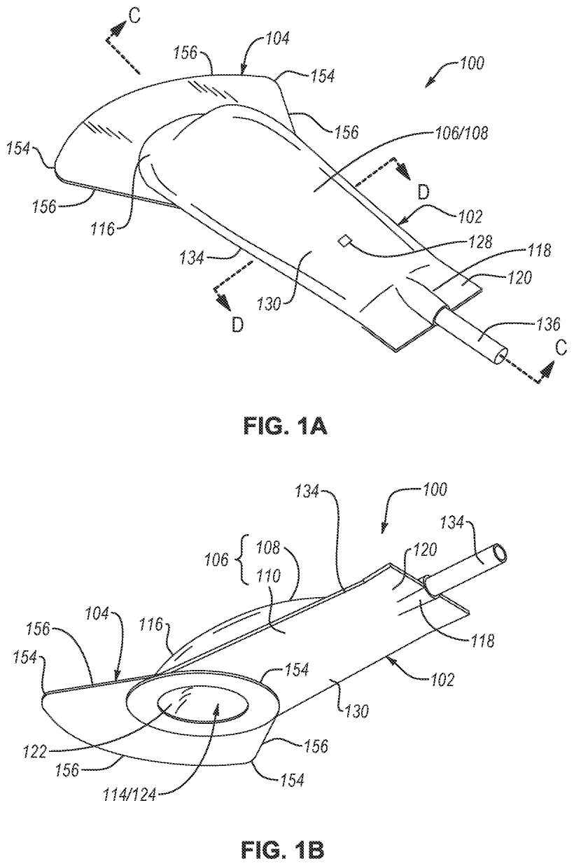

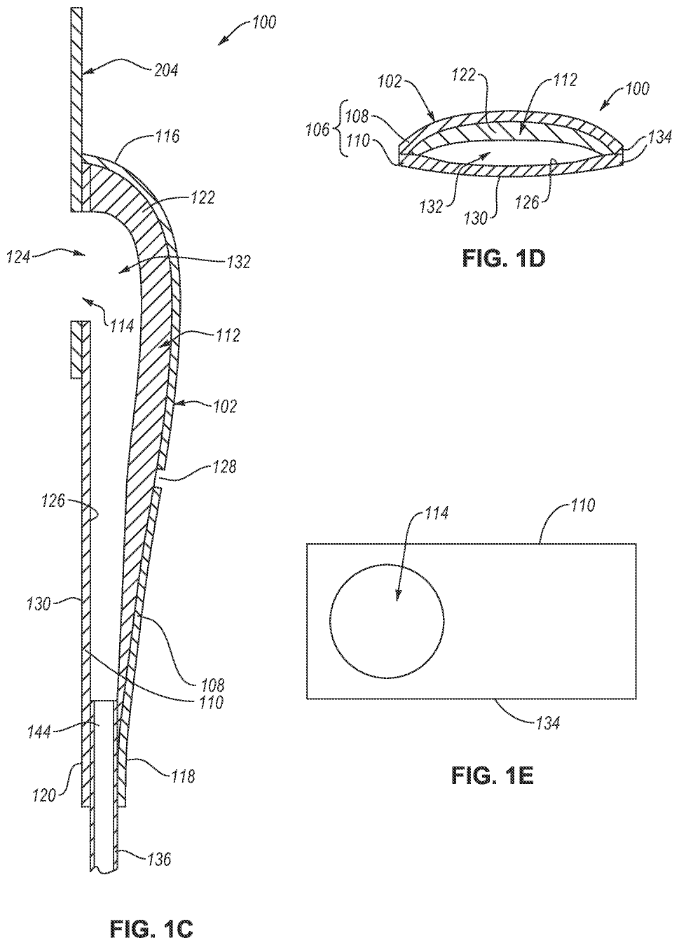

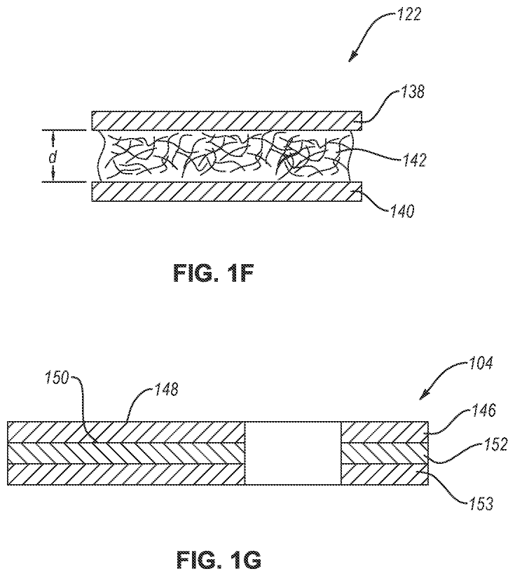

FIGS. 1A and 1B are isometric top and bottom views, respectively, of a fluid collection assembly 100, according to an embodiment. FIGS. 1C and 1D are cross-sectional schematics of the fluid collection assembly 100 taken along planes C-C and D-D, respectively, according to any embodiment. The fluid collection assembly 100 includes a sheath 102 and a base 104. The sheath 102 includes a fluid impermeable barrier 106 that is at least partially formed from a first panel 108 attached to a second panel 110. In an embodiment, as illustrated, the first panel 108 and the second panel 110 are distinct sheets. The fluid impermeable barrier 106 also defines a chamber 112 between the first panel 108 and the second panel, an opening 114 at a proximal end region 116 of the sheath 102, and an outlet 118 at a distal end region 120 of the sheath 102. The sheath 102 also includes at least one porous material 122 disposed in the chamber 112. The base 104 includes an aperture 124. The base 104 is permanently attached to the proximal end region 116 of the sheath 102 such that the aperture 124 is aligned with the opening 114. Permanently attached means that the sheath 102 cannot be detached from the base 104 without damaging at least one of the sheath 102 or the base 104, using a blade to separate the sheath 102 from the base 104, using chemicals to dissolve the adhesive that attaches the sheath 102 to the base 104, and/or using heat to melt or soften the adhesive or attachment (e.g., ultrasonic weld) that attached the sheath 102 to the base 104.

The inner surfaces 126 of the fluid impermeable barrier 106 (e.g., inner surfaces of the first and second panels 108, 110) at least partially defines the chamber 112 within the fluid collection assembly 100. The fluid impermeable barrier 106 temporarily stores the bodily fluids in the chamber 112. The fluid impermeable barrier 106 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 106 substantially prevents the bodily fluids from passing through the fluid impermeable barrier 106. In an example, the fluid impermeable barrier 106 may be air permeable and fluid impermeable thus preventing leaks while allowing air flow through the chamber 112 when a suction force is applied thereto (i.e., the chamber 112 remains at about atmospheric pressure thereby preventing the suction force from causing a hickie or kinking the conduit 136). In such an example, the fluid impermeable barrier 106 may be formed of a hydrophobic material that defines a plurality of pores. Alternatively or additionally, the fluid impermeable barrier 106 may include at least one perforation 128 (e.g., vacuum relief hole) that allows the chamber 112 to remain substantially at atmospheric pressure. At least one or more portions of at least an outer surface 130 of the fluid impermeable barrier 106 may be formed from a soft and/or smooth material, thereby reducing chaffing.

In an embodiment, at least one of the first panel 108 or the second panel 110 is formed from an at least partially transparent fluid impermeable material, such as polyethylene, polypropylene, polycarbonate, or polyvinyl chloride. Forming at least one of the first panel 108 or the second panel 110 from an at least partially transparent fluid impermeable material allows a person (e.g., medical practitioner) to examiner the penis. In some embodiments, both the first panel 108 and the second panel 110 are formed from at least partially transparent fluid impermeable material. For example, some conventional fluid collection assemblies that include a sheath and a base may allow the sheath to be reversibly detached from the base after the base is secured to the region about the penis. Detaching the sheath from the base allows the person to examine the penis. However, configuring the sheath to be detachable from the base may allow leaks between the sheath and the base. As previously discussed, the sheath 102 is permanently attached to the base 104 which substantially prevents leaks between the sheath 102 and the base 104 when the base 104 is appropriately attached to the sheath 102 (e.g., no wrinkles were allowed to form between the sheath 102 and base 104). Selecting at least one of the first panel 108 or the second panel 110 to be formed from an at least partially transparent impermeable material allows the penis to be examined without detaching the entire fluid collection assembly 100 from the region about the penis. For example, the chamber 112 may include a penis receiving area 132 that is configured to receive the penis of the individual when the penis extends into the chamber 112. The penis receiving area 132 may be defined by at least the porous material 122 and at least a portion of the at least partially transparent material of the first panel 108 and/or the second panel 110. In other words, the porous material 122 is positioned in the chamber 112 such that the porous material is not positioned between the penis and at least a portion of the transparent portion of the first panel 108 and/or second panel 110 when the penis is inserted into the chamber 112 through the opening 114 (e.g., the penis is positioned between at least a portion of the second panel and the at least one porous material). The porous material 122 is generally not transparent and, thus, the portion of the at least partially transparent material of the first panel 108 and/or the second panel 110 that defines the penis receiving area 132 forms a window which allows the person to view into the penis receiving area 132 and examine the penis.

In an embodiment, the second panel 110 is at least partially formed from the at least partially transparent material and forms the window that allows the person to view into the penis receiving area 132. Further, the porous material 122 is positioned between the penis receiving area 132 and at least a portion of the first panel 108. Such an embodiment may help maintain the dignity of the individual using the fluid collection assembly 100. For example, during use, the second panel 110 is generally adjacent to the individual, such as adjacent to the thighs and/or perineum. Thus, the second panel 110 is generally obscured during use and a person cannot view the penis without first lifting the sheath 102 away from the individual. Meanwhile, the first panel 108 may face away from the individual and be more easily viewable than the second panel 110. However, a person (e.g., a passerby, a visitor, etc.) cannot view the penis through the first panel 108 because the porous material 122 is not transparent and/or the first panel 108 is formed from a non-transparent material. Thus, in such an embodiment, the first panel 108 and/or the porous material 122 prevent person(s) from viewing the penis unless such examination is necessary, thereby preserving the dignity of the individual using the fluid collection assembly 100. In an embodiment, the first panel 108 is formed from the at least partially transparent material and forms the window that allows the person to view into the penis receiving area 132. Further, the porous material 122 is positioned between the penis receiving area 132 and at least a portion of the second panel 110. In such an embodiment, the person does not need to perform the additional act of lifting the sheath 102 to view into the penis receiving area 132 but may not maintain the dignity of the individual using the fluid collection assembly 100 since passersby may also view into the penis receiving area 132.

As previously discussed, at least a portion of the first panel 108 and at least a portion of the second panel 110 are attached together. In an embodiment, as shown, the first and second panels 108, 110 are attached together along at least a portion of the outer edges 134 thereof. In such an embodiment, the first and second panels 108, 110 are attached using any suitable technique, such as with an adhesive, sewing, heat sealing, radio frequency (“RF”) welding, ultrasonic (“US”) welding, or any other technique. As will be discussed in more detail below, forming the fluid impermeable barrier 106 from the first panel 108 and the second panel 110 may improve the rate of manufacturing the fluid collection assembly 100, especially when the first panel 108 and the second panel 110 are attached together using a non-sewing technique (e.g., the first and second panels 108, 110 are free from a sewn joint formed by sewing the first and second panels 108, 110 together).

The opening 114 defined by the fluid impermeable barrier 106 provides an ingress route for fluids to enter the chamber 112 when the penis is a buried penis and allow the penis to enter the chamber 112 (e.g., the penis receiving area 132) when the penis is not buried. The opening 114 may be defined by the fluid impermeable barrier 106 (e.g., an inner edge of the fluid impermeable barrier 106). For example, the opening 114 is formed in and extends through the fluid impermeable barrier 106, from the outer surface 130 to the inner surface 126, thereby enabling bodily fluids to enter the chamber 112 from outside of the fluid collection assembly 100.

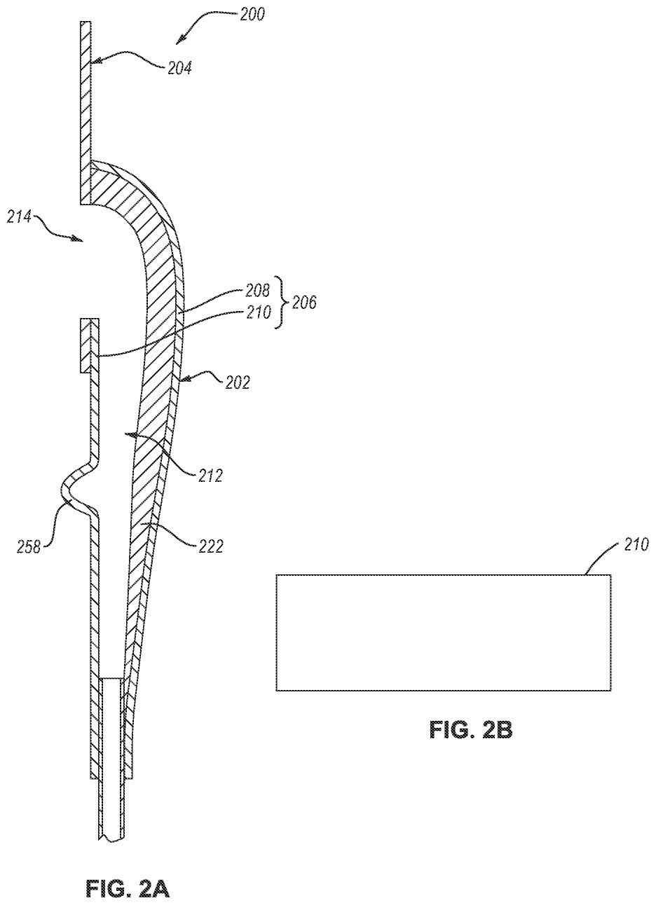

FIG. 1E is a top plan view of the second panel 110 of the fluid collection assembly 100, according to an embodiment. In the illustrated embodiment, the second panel 110 defines the entirety of the opening 114. For example, the opening 114 is a cutout defined by the second panel 110 that is spaced from the outer edges 134 of the second panel 110. In such an example, the second panel 110 may exhibit a shape that substantially corresponds to the shape of the first panel 108 which may facilitate attaching the first panel 108 to the second panel 110 along the outer edges thereof. It also allows the first panel 108 and the second panel 110 to lie substantially flat when the penis is not in the chamber 112 which may make wearing the fluid collection assembly 100 more discrete and inhibit pooling of bodily fluids against the individual. However, in some embodiments, the opening 114 is not spaced from the outer edges 134 of the second panel 110. In such embodiments, the opening 114 may be a cutout extending inwardly from at least one outer edge 134 of the second panel 110. Also, as will be discussed in more detail with regards to FIGS. 2A and 2B, the opening 114 may not be formed as a cutout.

Referring back to FIGS. 1A-1D, the fluid impermeable barrier 106 defines an outlet 118 sized to receive a conduit 136. The conduit 136 may be at least partially disposed in the chamber 112 or otherwise in fluid communication with the chamber 112 through the outlet 118. The outlet 118 may be sized and shaped to form an at least substantially fluid tight seal against the conduit 136 thereby substantially preventing the bodily fluids from escaping the chamber 112. In an embodiment, the outlet 118 may be formed from a portion of the first panel 108 and the second panel 110 that are not attached together. In such an embodiment, the fluid impermeable barrier 106 may not include a cap exhibiting a rigidity that is greater than the portions of the fluid impermeable barrier 106 thereabout which may facilitate manufacturing of the fluid collection assembly 100 may decreasing the number of parts that are used to form the fluid collection assembly 100 and may decrease the time required to manufacture the fluid collection assembly 100. The lack of the cap may make securing the conduit 136 to the outlet 118 using interference fit difficult though, it is noted, attaching the conduit 136 to the outlet 118 using interference fit may still be possible. As such, the conduit 136 may be attached to the outlet 118 (e.g., to the first and second panels 108, 110) using an adhesive, a weld, or otherwise bonding the outlet 118 to the outlet 118. Attaching the conduit 136 to the outlet 118 may prevent leaks and may prevent the conduit 136 from inadvertently becoming detached from the outlet 118. In an example, the conduit 136 may be attached to the outlet 118 in the same manufacturing step that attaches the first and second panels 108, 110 together.

As previously discussed, the sheath 102 includes at least one porous material 122 disposed in the chamber 112. The porous material 122 may direct the bodily fluids to one or more selected regions of the chamber 112, such as away from the penis and towards the outlet 118. As such, the porous material 122 may facilitate the removal of the bodily fluids from the chamber 112 and form a padding layer that prevents the penis from resting against a damp material which may cause degradation of the skin of the penis and/or make the fluid collection assembly 100 more uncomfortable to wear. The porous material 122 may also blunt a stream of urine from the penis.

In an embodiment, the porous material 122 is a wicking material configured to wick any bodily fluids away from the opening 114 thereby preventing the bodily fluids from escaping the chamber 112. Such “wicking” may not include absorption of fluid into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 30 wt % of the dry weight of the wicking material, less than 20 wt %, less than 15 wt %, less than 10 wt %, less than about 7 wt %, less than about 5 wt %, less than about 3 wt %, less than about 2 wt %, less than about 1 wt %, or less than about 0.5 wt % of the dry weight of the wicking material. The wicking material may also wick the fluid generally towards an interior of the chamber 112, as discussed in more detail below. In an embodiment, the porous material 122 is configured to adsorb or absorb the bodily fluids. Similar, to the wicking material, such adsorbing or absorbing material may move bodily fluids away from the opening 114 thereby preventing the bodily fluids from escaping the chamber 112.

The porous material 122 may be formed from any suitable porous material. For example, the porous material 122 may be formed from nylon (e.g., spun nylon fibers), polyester, polyurethane, polyethylene, polypropylene, other porous polymers, hydrophobic foam, an open cell foam, wool, silk, linen, cotton (e.g., cotton gauze), felt, other fabrics, a coated porous material (e.g., a water repellent coated porous material), any other suitable porous materials, or combinations thereof.

FIG. 1F is a cross-sectional schematic of a porous material 122, according to an embodiment. The porous material 122 includes a first layer 138 and a second layer 140. The first and second layers 138, 140 may be a woven material. The porous material 122 also includes a plurality of fibers 142 forming a layer between the first layer 138 and the second layer 140. Each of the first layer 138, the second layer 140, and the plurality of fibers 142 define a plurality of pores, thereby allowing transport of the bodily fluids and air circulation through the porous material 122. The pores defined by the plurality of fibers 142 may be at least one of larger or more numerous, thereby decreasing the likelihood that dried bodily fluids clog the porous material 122. The presence of the plurality of fibers 142 also cause the porous material 122 feel soft against the penis and provides a cushioning effect to the penis. The plurality of fibers 142 may also prevent the suction force from collapsing the porous material.

In an embodiment, the plurality of fibers may space the first layer 138 and the second layer 140 by a distance d. The distance d may be selected based on the number of fibers that form the plurality of fibers 142 and the density at which the plurality of fibers 142 are packed together. For example, the distance d may be selected to be about 0.25 mm or more, about 0.5 mm or more, about 0.75 mm or more, about 1 mm or more, about 1.5 mm or more, about 2 mm or more, about 3 mm or more, about 4 mm or more, about 5 mm or more, about 6 mm or more, about 8 mm or more, about 10 mm or more, about 12.5 mm or more, about 15 mm or more, or in ranges of about 0.25 mm to about 0.75 mm, about 0.5 mm to about 1 mm, about 0.75 mm to about 1.5 mm, about 1 mm to about 2 mm, about 1.5 mm to about 3 mm, about 2 mm to about 4 mm, about 3 mm to about 5 mm, about 4 mm to about 6 mm, about 5 mm to about 8 mm, about 6 mm to about 10 mm, about 8 mm to about 12.5 mm, or about 10 mm to about 15 mm. The thickness of the distance d may be selected to adjust the absorbency of the porous material 122. For example, increasing the thickness may increase the volume of the plurality of fibers 142 and/or the porosity defined by the plurality of fibers 142 which increases the amount of bodily fluids that may be received by and at least partially stored in the porous material 122.

The first layer 138, the second layer 140, and the plurality of fibers 142 may be formed from any suitable material, such as a hydrophobic material, a hydrophilic material, polyester, cotton, or any other porous material disclosed herein. In an embodiment, one or more of the first layer 138, the second layer 140, or the plurality of fibers 142 are formed from a hydrophobic material that inhibits the porous material 122 from storing the bodily fluids therein which may facilitate removal of the bodily fluids from the chamber 112. In an embodiment, one or more of the first layer 138, and second layer 140, or the plurality of fibers 142 are formed from a hydrophilic material which allows the porous material 122 to temporarily store the bodily fluids therein thereby limiting the quantity of bodily fluids that pool around the skin of the individual. In an embodiment, two or more of the first layer 138, the second layer 140, or the plurality of fibers 142 are formed from different materials. In such an embodiment, the first layer 138 may define the penis receiving area 132 or is otherwise closer to the penis receiving area 132 than the second layer 140. The first layer 138 may be formed from a hydrophobic material while the plurality of fibers 142 are formed from a hydrophilic material. Such a configuration may cause the bodily fluids to be pulled through the first layer 138 and temporarily stored in the plurality of fibers 142. However, the first layer 138 may remain substantially dry due to the hydrophobicity thereof which allows the porous material 122 to feel dry to the penis.

In an embodiment, not shown, the porous material 122 may be formed from two layers instead of the three layers illustrated in FIG. 1F. For example, the porous material 122 may be formed from a fluid permeable membrane and a fluid permeable support. The fluid permeable support may define or otherwise be closer to the penis receiving area 132 than the fluid permeable support. The fluid permeable membrane may be composed and/or structured to wick bodily fluids away from the penis receiving area 132, thereby minimizing the quantity of bodily fluids that are present in the penis receiving area 132 or otherwise present against the skin of the individual. It is also noted that the fluid permeable membrane may also be configured to adsorb or absorb the bodily fluids to minimize the quantity of bodily fluids that are present in the penis receiving area 132 or otherwise present against the skin of the individual. The fluid permeable membrane may be formed from any of the porous materials disclosed herein. For example, the fluid permeable membrane may be formed from fabric, such as a gauze (e.g., silk, linen, or cotton gauze), another soft fabric, or another smooth fabric. Forming the fluid permeable membrane from gauze, soft fabric, and/or smooth fabric (or any of the other porous materials 122 disclosed herein that may contact the penis) may reduce chaffing caused by the fluid collection assembly 100.

The fluid permeable support is configured to support the fluid permeable membrane since the fluid permeable membrane may be formed from a relatively foldable, flimsy, or otherwise easily deformable material. For example, the fluid permeable support may be positioned such that the fluid permeable membrane is disposed between the fluid permeable support and the fluid impermeable barrier 106. As such, the fluid permeable support may support and maintain the position of the fluid permeable membrane. The fluid permeable support may include any of the fluid permeable membrane materials disclosed herein above. For example, the fluid permeable membrane material(s) may be utilized in a more dense or rigid form than in the fluid permeable membrane when used as the fluid permeable support. The fluid permeable support may be formed from any fluid permeable material that is less deformable than the fluid permeable membrane. For example, the fluid permeable support may include a porous polymer (e.g., nylon, polyester, polyurethane, polyethylene, polypropylene, etc.) structure or an open cell foam. In some examples, the fluid permeable support may be formed from a natural material, such as cotton, wool, silk, or combinations thereof. In such examples, the material may have a coating to prevent or limit absorption of fluid into the material, such as a water repellent coating. In some examples, the fluid permeable support may be formed from fabric, felt, gauze, or combinations thereof.

In an embodiment, the porous material 122 may include a single layer (e.g., one of the first layer 138, the second layer 140, the layer formed from the plurality of fibers 142, the fluid permeable membrane, the fluid permeable support, or another porous layer). In an embodiment, the porous material 122 may be formed from four or more layers.

In an embodiment, the porous material 122 may be a sheet (e.g., a multi-layer sheet). The porous material 112 is a sheet when the porous material 112 at least one of is generally planar when lying on a flat surface, does not define a cavity (e.g., is not tubular), or exhibits a length and width that is greater than a thickness thereof. Forming the porous material 122 as a sheet may facilitate the manufacturing of the fluid collection assembly 100. For example, forming the porous material 122 as a sheet allows the first panel 108, the second panel 110, and the porous material 122 to each be sheets. During the manufacturing of the fluid collection assembly 100, the first panel 108, the second panel 110, and the porous material 122 may be stacked and then attached to each other in the same manufacturing step. For instance, the porous material 122 may exhibit a shape that is the same size or, more preferably, slightly smaller than the size of the first panel 108 and the second panel 110. As such, attaching the first panel 108 and the second panel 110 together along the outer edges 134 thereof may also attach the porous material 122 to the first panel 108 and the second panel 110. The porous material 122 may be slightly smaller than the first panel 108 and the second panel 110 such that the first panel 108 and/or the second panel 110 extend around the porous material 122 such that the porous material 122 does not form a passageway through the fluid impermeable barrier 106 through which the bodily fluids may leak. Also, attaching the porous material 122 to the first panel 108 and/or the second panel 110 may prevent the porous material 122 from significantly moving in the chamber 112, such as preventing the porous material 122 from bunching together near the outlet 118. In an example, the porous material 122 may be attached to the first panel 108 or the second panel 110 (e.g., via an adhesive) before or after attaching the first panel 108 to the second panel 110. In an example, the porous material 122 may merely be disposed in the chamber 112 without attaching the porous material 122 to at least one of the first panel 108 or the second panel 110. In an embodiment, as will be discussed in more detail below, the porous material 122 may exhibit shapes other than a sheet, such as a hollow generally cylindrical shape.

Generally, the sheath 102 is substantially flat when the penis is not in the penis receiving area 132 and the sheath 102 is resting on a flat surface. The sheath 102 is substantially flat because the fluid impermeable barrier 106 is formed from the first panel 108 and the second panel 110 instead of a generally tubular fluid impermeable barrier. Further, as previously discussed, the porous material 122 may be a sheet, which also causes the sheath 102 to be substantially flat. The sheath 102 may also be substantially flat because the fluid collection assembly 100 may not include relatively rigid rings or caps that exhibit a rigidity that is greater than the portions of the fluid impermeable barrier 106 thereabout since such rings and caps may inhibit the sheath 102 being substantially flat. In other words, the sheath 102 may be free from the one or more ring and/or the cap. It is noted that the sheath 102 is described as being substantially flat because at least one of the porous material 122 may cause a slight bulge to form in the sheath 102 depending on the thickness of the porous material 122, the outlet 118 and/or conduit 136 may cause a bulge thereabout, or the base 104 may pull on portions of the sheath 102 thereabout. It is also noted that the sheath 102 may also be compliant and, as such, the sheath 102 may not be substantially flat during use since, during use, the sheath 102 may rest on a non-flat surface (e.g., may rest on the testicles, the perineum, and/or between the thighs) and the sheath 102 may conform to the surface of these shapes.

The ability of the sheath 102 to be substantially flat when the penis is not in the penis receiving area 132 and the sheath 102 is resting on a flat surface allows the fluid collection assembly 100 to be used with a buried and a non-buried penis. For example, when the fluid collection assembly 100 is being used with a buried penis, the penis does not extend into the penis receiving area 132 which causes the sheath 102 to lie relatively flat across the aperature 124. When the sheath 102 lies relatively flat across the aperture 124, the porous material 122 extends across the aperature and is in close proximity to the buried penis. As such, the porous material 122 prevents or inhibits pooling of bodily fluids discharged from the buried penis against the skin of the individual since the porous material 122 will receive and remove at least a significant portion of the bodily fluids that would otherwise pool against the skin of the individual. Thus, the skin of the individual remains dry thereby improving comfort of using the fluid collection assembly 100 and preventing skin degradation. However, unlike other conventional fluid collection assemblies that are configured to be used with buried penises, the fluid collection assembly 100 may still be used with a non-buried penis since the non-buried penis can still be received into the penis receiving area 132, even when the penis is fully erect. Additionally, the ability of the sheath 102 to be substantially flat allows the fluid collection assembly 100 to be used more discretely than if the sheath 102 was not substantially flat thereby avoiding possibly embarrassing scenarios.

When the sheath 102 is substantially flat, the porous material 122 occupies substantially all of the chamber 112 and the penis receiving area 132 is collapsed (shown as being non-collapsed in FIGS. 1C and 1D for illustrative purposes). In other words, the sheath 102 may not define an region that is constantly unoccupied by the porous material 122. When the porous material 122 occupies substantially all of the chamber 112, the bodily fluids discharged into the chamber 112 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 108, the second panel 110, and the porous material 122 may be selected to be relatively flexible. The first panel 108, the second panel 110, and the porous material 122 are relatively flexible when the first panel 108, the second panel 110, and the porous material 122, respectively, are unable to maintain their shape when unsupported. The flexibility of the first panel 108, the second panel 110, and the porous material 122 may allow the sheath 102 to be substantially flat, as discussed above. The flexibility of the first panel 108, the second panel 110, and the porous material 122 may also allow the sheath 102 to conform to the shape of the penis even when the size and shape of the penis changes (e.g., becomes erect) and to minimize any unoccupied spaces in the chamber 112 in which bodily fluids may pool.

As previously discussed, the fluid collection assembly 100 includes a base 104 that is configured to be permanently attached to the sheath 102. The base is configured to be permanently attached to the sheath 102 when, for example, the fluid collection assembly 100 is provided with the base 104 permanently attached to the sheath 102 or the base 104 is provided without being permanently attached to the sheath 102 but is configured to be permanently attached to the sheath 102 at some point in the future. The base 104 may be permanently attached to the sheath 102 using any suitable technique. For example, the base 104 may be permanently attached to the sheath 102 using an adhesive, sewing, heat sealing, RF welding, or US welding.

As previously discussed, the base 104 is sized, shaped, and made of a material to be coupled to the skin that surrounds the penis (e.g., mons pubis, thighs, testicles, and/or perineum) and have the penis disposed therethrough. For example, the base 104 may define an aperture 124 configured to have the penis positioned therethrough. In an example, the base 104 may exhibit the general shape or contours of the skin surface that the base 104 is configured to be coupled with. The base 104 may be flexible, thereby allowing the base 104 to conform to any shape of the skin surface and mitigate the base 104 pulling the on skin surface. The base 104 may extend laterally past the sheath 102 thereby increasing the surface area of the skin of the individual to which the fluid collection assembly 100 may be attached compared to a substantially similar fluid collection assembly 100 that did not include a base.

FIG. 1G is a cross-sectional schematic of the base 104, according to an embodiment. The base 104 includes a substrate 146 having a top surface 148 and a bottom surface 150. The top surface 148 is closer to the sheath 102 than the bottom surface 150 while the bottom surface 150 is closer to the skin of the individual than the top surface 148. The base 104 may also include an adhesive layer 152 disposed on at least a portion of the bottom surface 150. The adhesive layer 152 is configured to attach the base 104 to the skin around the penis. The base 104 may also include a release liner 153 is configured to be easily removed from the adhesive layer 152 and is configured to prevent the adhesive layer 152 inadvertently becoming attached to an object.

The substrate 146 may be formed from a fluid impermeable material to prevent bodily fluids from leaking from the chamber 112 through the base 104. For example, the substrate 146 may be formed from any of the fluid impermeable materials disclosed herein.

In an embodiment, the substrate 146 is formed from a thin film exhibiting a thickness that is about 2 mm or less, about 1.5 mm or less, about 1 mm or less, about 0.75 mm or less, about 0.5 mm or less, about 0.3 mm or less, about 0.2 mm or less, about 0.1 mm or less, about 0.05 mm or less, or in ranges of about 0.05 mm to about 0.2 mm, about 0.1 mm to about 0.3 mm, about 0.2 mm to about 0.5 mm, about 0.3 mm to about 0.75 mm, about 0.5 mm to about 1 mm, about 0.75 mm to about 1.5 mm, or about 1 to about 2 mm. Forming the substrate 146 as a thin film exhibiting any of the above thicknesses may cause the substrate 146 to be sufficiently flexible to conform to the shape and contours of the skin surrounding the penis. For instance, the shape and contours of the skin surrounding the penis may vary from individual to individual and configuring the substrate 146 to be a thin film may allow the base 104 to conform to the shape and contour of the skin surrounding the penis while preventing the formation of any gaps between the base 104 and the skin through which bodily fluids may leak. Further, the thin film substrate 146 is able to be attached to the skin of the individual without or substantially without pulling the skin of the individual which makes the fluid collection assembly 100 more comfortable to use for prolonged periods of time. It is noted that the amount of the fluid collection assembly 100 pulls on the skin decreases with decreasing thickness of the substrate such that, for example, a substrate exhibiting a thickness of about 2 mm pulls more on the skin than a substantially similar substrate exhibiting a thickness of about 0.5 mm.

The adhesive layer 152 may be formed from any adhesive that may safely attach the substrate 146 to the skin surrounding the penis. In an example, the adhesive layer 152 may be formed from a silicone-based adhesive, such as a silicone-gel adhesive. Silicone-based adhesives, such as Silicone Medical Silicone Tape 2475P available from 3M, has been found to secure the fluid collection assembly 100 to the skin surrounding the penis for at least 24 hours, even immediately after cleaning the skin surface with a wipe. In an example, the adhesive layer 152 may be formed from an acrylic gel adhesive or a hydrogel.

In an embodiment, the base 104 is at least partially transparent (e.g., the substrate 146 and the adhesive layer 152 are formed from at least partially transparent materials). In such an embodiment, a person (e.g., medical practitioner) may be able to examiner the skin surrounding the penis, such as to determine the health of the skin. Further, the person may be able to detect any gaps between the base 104 and the skin of the individual through which bodily fluids may leak. A person may be able to eliminate the gaps or replace the fluid collection assembly 100 after detecting the gaps to prevent leaks and prevent degradation of the skin caused by the skin being in contact with the bodily fluids.

Referring back to FIGS. 1A-1D, in an embodiment, the base 104 may exhibit a generally partially triangular shape, as illustrated. For example, the base 104 may exhibit three apexes 154 and edges 156 extending between each of the apexes 154. The apexes 154 may be rounded to prevent the base 104 from digging into and hurting the individual. In an embodiment, the aperture 124 may be located off-center and closer to one of the apexes 154 than the other apexes 154. Such an embodiment may maximize the surface area of the skin to which the base 104 is attached, thereby reducing the likelihood that the base 104 leaks or inadvertently becomes detached from the individual. For example, the apex 154 closest the aperture 124 may be configured to be attached to the skin between the penis and the testicles and/or the testicles which has limited surface area to be attached to the base 104 and/or may be sensitive. Meanwhile, the portion of the base 104 opposite the apex 154 that is closest to the aperture 124 is configured to be attached to the mons pubis which has a large surface area and is less sensitive. Other examples of shapes that the base 104 may form are disclosed in PCT Application No. PCT/US2021/015787 filed on Jan. 29, 2021, the disclosure of which is incorporated herein, in its entirety, by this reference.

As previously discussed, the fluid collection assembly 100 includes a conduit 136. The conduit 136 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 136 may include silicon or latex. In some examples, the conduit 136 may include one or more portions that are resilient, such as to by having one or more of a diameter or wall thickness that allows the conduit to be flexible.

An inlet 144 of the conduit 136 may be located at or near the distal end region 120 of the sheath 102 which is expected to be the gravimetrically low point of the chamber 112 when worn by a user. Locating the inlet 144 at or near the distal end region 120 of the sheath 102 enables the conduit 136 to receive more of the bodily fluids than if the inlet 144 was located elsewhere and reduce the likelihood of pooling (e.g., pooling of the bodily fluids may cause microbe growth and foul odors). For instance, the bodily fluids in porous material 122 due to capillary forces. However, the bodily fluids may exhibit a preference to flow in the direction of gravity, especially when at least a portion of the porous material 122 is saturated with the bodily fluids. Accordingly, the inlet 144 may be located in the fluid collection assembly 100 in a position expected to be the gravimetrically low point in the fluid collection assembly 100 when worn by a user.

In an example, the conduit 136 is configured to be at least insertable into the chamber 112, such as into the penis receiving area 132. In such an example, the conduit 136 may include one or more markers (not shown) on an exterior thereof that are located to facilitate insertion of the conduit 136 into the chamber 112. For example, the conduit 136 may include one or more markings thereon that are configured to prevent over or under insertion of the conduit 136. In another example, the conduit 136 may include one or more markings thereon that are configured to facilitate correct rotation of the conduit 136 relative to the chamber 112. The one or more markings may include a line, a dot, a sticker, or any other suitable marking.

As described in more detail below, the conduit 136 is configured to be coupled to, and at least partially extend between, one or more of the fluid storage container (not shown) and the vacuum source (not shown). In an example, the conduit 136 is configured to be directly connected to the vacuum source (not shown). In such an example, the conduit 136 may extend from the fluid impermeable barrier 106 by at least one foot, at least two feet, at least three feet, or at least six feet. In another example, the conduit 136 is configured to be indirectly connected to at least one of the fluid storage container (not shown) and the vacuum source (not shown). In some examples, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

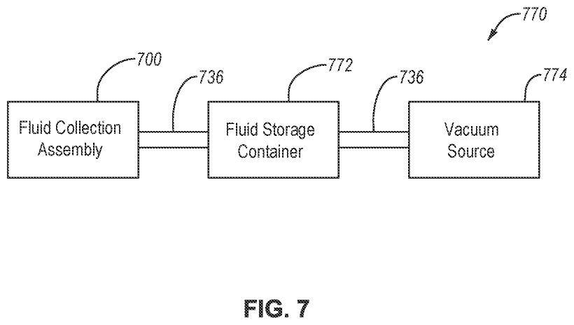

The inlet 144 and an outlet of the conduit 136 are configured to fluidly couple (e.g., directly or indirectly) the vacuum source (not shown) to the chamber 112. As the vacuum source (FIG. 7) applies a vacuum/suction in the conduit 136, the bodily fluids in the chamber 112 may be drawn into the inlet 144 and out of the fluid collection assembly 100 via the conduit 136. In some examples, the conduit 136 may be frosted or opaque (e.g., black) to obscure visibility of the bodily fluids therein.

In some examples, the vacuum source may be remotely located from the fluid collection device. In such examples, the conduit 136 may be fluidly connected to the fluid storage container, which may be disposed between the vacuum source and the fluid collection assembly 100.

During operation, a male using the fluid collection assembly 100 may discharge bodily fluids (e.g., urine) into the chamber 112. The bodily fluids may pool or otherwise be collected in the chamber 112 (e.g., received into the porous material 122). At least some of the bodily fluids may be pulled through the interior of the conduit 136 via the inlet 144. The bodily fluids may be drawn out of the fluid collection assembly 100 via the vacuum/suction provided by the vacuum source. During operation, the perforation 128 may substantially maintain the pressure in the chamber 112 at atmospheric pressure even though bodily fluids are introduced into and subsequently removed from the chamber 122.

FIG. 2A is cross-sectional schematic of a fluid collection assembly 200, according to an embodiment. Except as otherwise disclosed herein, the fluid collection assembly 200 is the same or similar to any of the other fluid collection assemblies disclosed herein, in one or more aspects. For example, the fluid collection assembly 200 includes a sheath 202 and a base 204. The sheath 202 includes a fluid impermeable barrier 206 that includes a first panel 208 and a second panel 210. The fluid impermeable barrier 206 may define a chamber 212 and an opening 214. The sheath 202 also includes at least one porous material 222 position in the chamber 212.

FIG. 2B is a top plan view of the second panel 210, according to an embodiment. As shown, the second panel 210 does not include a cutout similar to the second panel 110 shown in FIG. 1E, according to an embodiment. Instead, the second panel 210 exhibits a size and shape that is substantially similar to the first panel 208. When the second panel 210 does not include a cutout, both the first and second panels 208, 210 define the opening 214 However, referring back to FIG. 2A, the second panel 210 may form one or more wrinkle 258 when the fluid collection assembly 200 lies flat or is in use. The wrinkle 258 may slightly press against the individual, which may help maintain the position of the sheath 202 against the individual. However, manufacturing of the fluid collection assembly 200 may be simplified when the first panel 208 and the second panel 210 exhibit the same size and shape since only one type of panel is required to form the fluid collection assembly 200 instead of two.

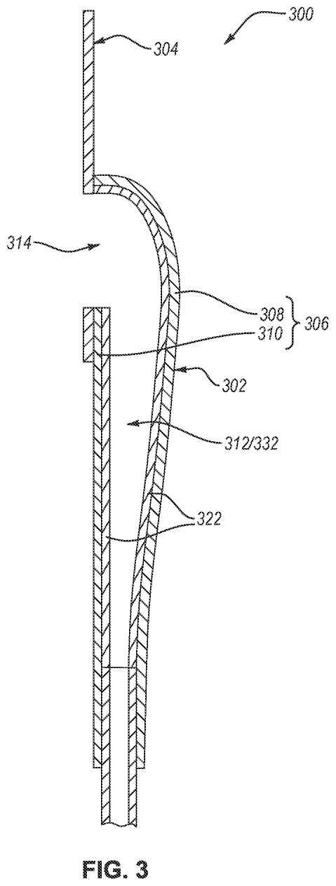

As previously discussed, the porous material of the fluid collection assemblies disclosed herein may exhibit a non-sheet like shape. FIG. 3 is a cross-sectional schematic of a fluid collection assembly 300, according to an embodiment. Except as otherwise disclosed herein, the fluid collection assembly 300 is the same or similar to any of the other fluid collection assemblies disclosed herein, in one or more aspects. For example, the fluid collection assembly 300 includes a sheath 302 and a base 304. The sheath 302 includes a fluid impermeable barrier 306 that includes a first panel 308 and a second panel 310. The fluid impermeable barrier 306 may define a chamber 312 and an opening 314. The sheath 302 also includes at least one porous material 322 position in the chamber 312.

The porous material 322 exhibits a non-sheet like shape. Examples non-sheet like shapes that the porous material 322 may exhibit includes a hollow generally cylindrical shape or at least two sheets that are attached together along at least a portion of the outer edges thereof. In an example, when the porous material 322 exhibits a non-sheet like shape, the porous material 322 may at least partially defined the penis receiving area 332 such that the porous material 322 is positioned between the penis receiving area 332 and at least a portion of the first panel 308 and between the penis receiving area 332 and at least a portion of the second panel 310. In such an example, the porous material 322 may prevent pooling of bodily fluids against portions of the first panel 308 or second panel 310 that would otherwise define the penis receiving area 332. However, the non-sheet like shape of the porous material 322 may cause the porous material 322 to be spaced from the opening 314. Further, the non-sheet like shape of the porous material 322 may prevent a person from viewing the penis in the penis receiving area 332, though this may be mitigated by forming one or more cutouts (not shown) in the porous material 322.

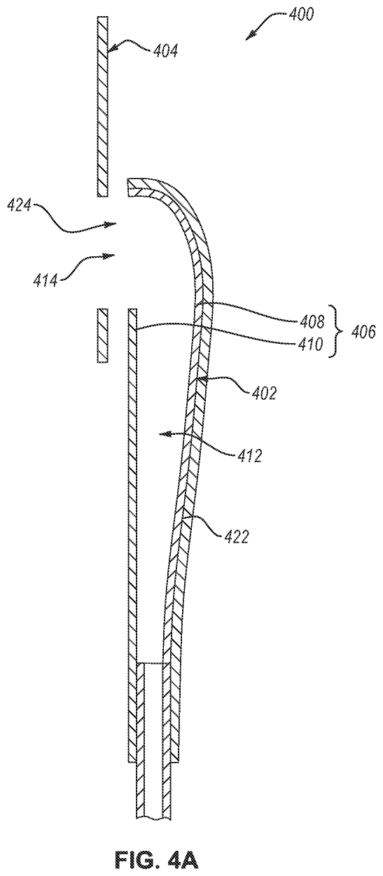

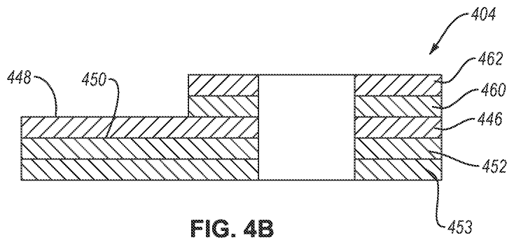

As previously discussed, the fluid collection assemblies disclosed herein may be provided with the base not attached to the sheath. Instead, the base may be configured to be attached to the sheath at some time after providing the fluid collection assembly. FIG. 4A is a cross-sectional schematic of a fluid collection assembly 400, according to an embodiment. Except as otherwise disclosed herein, the fluid collection assembly 400 is the same or similar to any of the other fluid collection assemblies disclosed herein, in one or more aspects. For example, the fluid collection assembly 400 includes a sheath 402 and a base 404. The sheath 402 includes a fluid impermeable barrier 406 that includes a first panel 408 and a second panel 410. The fluid impermeable barrier 406 may define a chamber 412 and an opening 414. The sheath 402 also includes at least one porous material 422 position in the chamber 412.

The base 404 is not permanently attached to the sheath 402. Instead, the base 404 may be permanently attached to the sheath 402 before, during, or after attaching the base 404 to the skin surrounding the penis. In an example, attaching the base 404 to the sheath 402 after providing the fluid collection assembly 400 may allow for a base to be selected from a plurality of differently shaped and/or contoured bases. As previously discussed, the shape and contours of the skin surrounding the penis may vary from individual to individual. Selecting a base from the plurality of bases allows a base to be selected at least one of having the maximum possible size to limit leaks and improve the attachment between the base and the skin or exhibiting a contour that better corresponds to the contour of the skin to limit pulling. In an example, a person applying the base 404 to the individual may find it easier to correctly position the base 404 on the individual when the sheath 402 is not attached to the base 404 since not attaching the sheath 402 to the base 404 allows the person to more easily see through the aperture 424. In an embodiment, providing the fluid collection assembly 400 with the base 404 not attached to the sheath 402 may allow the sheath 402 to be attached to the base 404 depending on the position of the individual. For instance, the angle at which the sheath 402 extends from the base may vary depending on whether the individual is laying on the individual's back or side.

FIG. 4B is a cross-sectional schematic of the base 404 shown in FIG. 4A, according to an embodiment. Except as otherwise disclosed herein, the base 404 may be substantially similar to any of the bases disclosed herein, such as the base 104 illustrated in FIG. 1F. For example, the base 404 may include a substrate 446 defining a top surface 448 and a bottom surface 450. The base 404 may include a first adhesive layer 452 attached to at least a portion of the bottom surface 450 and a first release liner 453 attached to the first adhesive layer 452. The first release liner 453 is configured to be easily removed from the first adhesive layer 452 and is configured to prevent the first adhesive layer 452 inadvertently becoming attached to an object.

The base 404 also include a second adhesive layer 460 on at least a portion of the top surface 448 of the substrate 446. Generally, the second adhesive layer 460 is disposed on, at most, the portions of the top surface 448 that are adjacent to the sheath 402 after the sheath 402 is attached to the base 404 thereby preventing a portion of the second adhesive layer 460 being exposed after attaching the sheath 402 to the base 404. The second adhesive layer 460 may include any of the adhesives disclosed herein or any other suitable adhesive and is configured to form the permanent attachment between the sheath 402 and the base 404. The base 404 may also include a second release liner 462 disposed on the second adhesive layer 460. The second release liner 462 is configured to be easily removed from the second adhesive layer 460 and is configured to prevent the second adhesive layer 460 inadvertently becoming attached to an object.

It is noted that, in some embodiments, the second adhesive layer 460 and the second release liner 462 may be omitted from the base 404. For example, the base 404 may be configured to be permanently attached to the sheath 402 using tape (e.g., double-sided tape), glue, or any other suitable attachment technique.

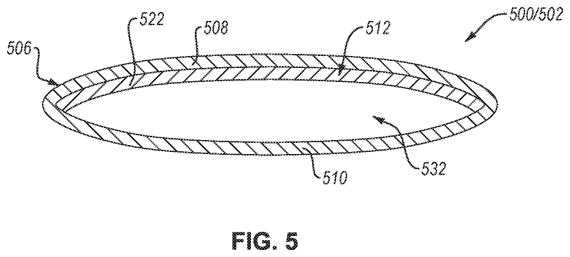

The embodiments of fluid collection assemblies disclosed above are formed from a first panel and a second panel that are distinct sheets. As previously discussed, forming the first panel and the second panel from distinct sheets may allow the first panel to be at least partially opaque while the second panel is at least partially transparent. Thus, the first panel may obstruct viewing the penis and whether there are bodily fluids in the chamber thereby increasing patient privacy while the second panel allows examination of the penis. However, forming the first and second panels from distinct sheets may form edges that may cause patient discomfort and requires a significant amount of manufacturing (e.g., welding or other attachment technique) to attach the first panel to the second panel. As such, in other embodiments, the fluid collection assemblies disclosed herein may be formed from first and second panels that are integrally formed together (e.g., exhibit single piece construction) which may eliminate at least some of the edges and simplify manufacturing of such fluid collection assemblies. For example, FIG. 5 is a cross-sectional schematic view of a fluid collection assembly 500 that includes a first panel 508 and a second panel 510 integrally formed together, according to an embodiment. Except as otherwise disclosed herein, the fluid collection assembly 500 may be the same or substantially similar to any of the fluid collection assemblies disclosed herein. For example, the fluid collection assembly 500 includes a sheath 502 and a base (not shown). The sheath 502 at least defines a chamber 512, an opening (not shown), and a fluid outlet (not shown). The fluid collection assembly 500 also includes at least one porous material 522 disposed in the chamber 512.

As previously discussed, the first panel 508 and the second panel 510 are integrally formed together. As such, the first panel 508 and the second panel 510 are different regions of fluid impermeable barrier 502 instead of different sheets that are attached together. The first panel 508 is the region of the fluid impermeable barrier 502 adjacent to the porous material 522 while the second panel 510 is the region of the fluid impermeable barrier 502 adjacent to the penis receiving area 532 (shown in a non-collapsed state for illustrative purposes).

In an embodiment, as illustrated, the first and second panels 508, 510 are formed from a thin walled tube, such as thin walled tube formed using a blown film extrusion process. Forming the first and second panels 508, 510 from the thin walled tube precludes the need to attach the longitudinal edges of the first and second panels 508, 510 together. However, the portions of the first and second panels 508, 510 that form the proximal end region and the distal end region thereof may need to be attached together using any of the techniques disclosed herein. As such, forming the first and second panels 508, 510 from a thin walled tube makes manufacturing of the fluid collection assembly 500 more efficient since fewer portions of the first and second panels 508, 510 need to be attached together. Further, forming the first and second panels 508, 510 from the thin walled tube decreases the number of edges that may press into the individual compared to forming the first and second panels 508, 510 from two distinct sheets.

In an embodiment, the first and second panels 508, 510 are formed from a single sheet that is folded. Forming the first and second panels 508, 510 from the single folded sheet precludes the need to attach one of the longitudinal edges of the first and second panels 508, 510 together. However, the portions of the first and second panels 508, 510 that form the proximal end region, the distal end region, and the portion opposite the fold may need to be attached together using any of the attachment techniques disclosed herein. Thus, forming the first and second panels 508, 510 from a single folded sheet makes manufacturing of the fluid collection assembly 500 more efficient and decreases the number of edges that are formed compared to forming the first and second panels 508, 510 from two distinct sheets. It is noted that, in either embodiment, the first panel 508 and the second panel 510 may still lie substantially flat when the penis is not in the chamber 512.

In an embodiment, the second panel 510 may have an opening formed therein. For example, the second panel 510 may have a cutout formed therein that is spaced from or extends inwardly from an outer edge of the second panel 510 (e.g., an outer edge of the thin walled tube or the single folded sheet). In such an example, the opening may be substantially similar to the opening 114 of FIGS. 1B, 1C, and 1E. In an embodiment, a portion of the edges of the first and second panels 508, 510 at the proximal end region thereof are not attached together and first and second panels 508, 510 collectively define the opening. In such an embodiment, the opening may be substantially similar to the opening 214 shown in FIG. 2A.

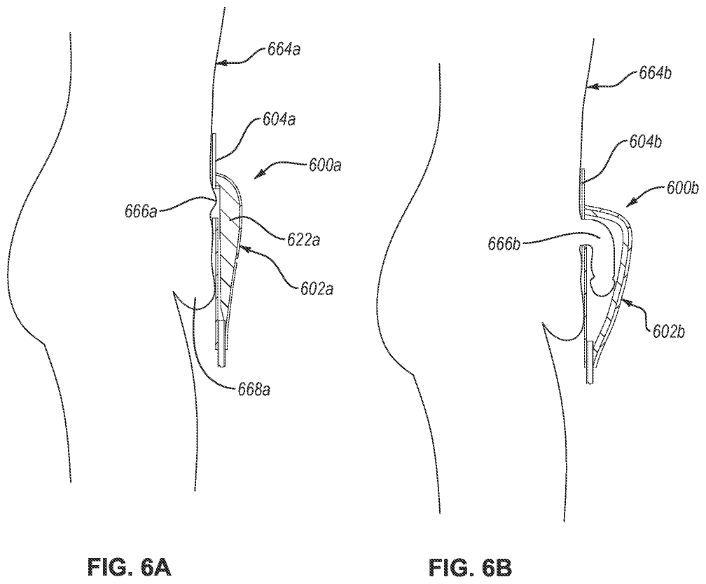

As previously discussed, the fluid collection assemblies disclosed herein may be used with a buried penis and a non-buried penis. FIGS. 6A and 6B are cross-sectional schematics illustrating how substantially similar fluid collection assemblies may be used with a buried and non-buried penis, according to an embodiment. Referring to FIG. 6A, a first individual 664a has a buried penis 666a (shown schematically as a slight bump). The fluid collection assembly 600a is attached to the first individual 664a. The fluid collection assembly 600 is illustrated as being substantially the same as fluid collection 25 assembly 100 shown in FIGS. 1A-1D. However, it is noted that the fluid collection assembly 600a may include any of the fluid collection assemblies disclosed herein. The fluid collection assembly 600a is attached to the first individual 664a such that the aperture of the base 604a and the opening of the sheath 602a is adjacent to the buried penis 666a. Since the fluid collection assembly 600a lies substantially flat, the porous material 622a is generally adjacent to the opening and may only be spaced from the buried penis 666a by the thickness of the base 604a which prevents almost no space for the bodily fluids to pool before being received by the porous material 622a. Is noted that the porous material 622a may be spaced from the buried penis 666a by a distance that is less than the thickness of the base 604a (e.g., the porous material 622a bulges into the aperture) or slightly greater than the thickness of the base 604a (e.g., the testicles 668a cause a bulge that pushes the porous material 622a away from the opening 614a or the porous material 622a exhibits a non-sheet like shape).

Referring to FIG. 6B, a second individual 664b has a non-buried penis 666b. A fluid collection assembly 600b that is the same or substantially the same as the fluid collection assembly 600a is attached to the second individual 664b. The fluid collection assembly 600b is attached to the second individual 664b such that the penis 656b extends through the aperture of the base 604b and through the opening of the sheath 602b. Due to the flexibility of the fluid collection assembly 600b, the sheath 602b can receive the penis therein.

FIG. 7 is a block diagram of a system 770 for fluid collection, according to an embodiment. The system 770 includes a fluid collection assembly 700, a fluid storage container 772, and a vacuum source 774. The fluid collection assembly 700, the fluid storage container 772, and the vacuum source 774 may be fluidly coupled to each other via one or more conduits 736. For example, fluid collection assembly 700 may be operably coupled to one or more of the fluid storage container 772 or the vacuum source 774 via the conduit 736. Bodily fluids (e.g., urine or other bodily fluids) collected in the fluid collection assembly 700 may be removed from the fluid collection assembly 700 via the conduit 736 which protrudes into the fluid collection assembly 700. Suction force may be introduced into the chamber of the fluid collection assembly 700 via the inlet of the conduit 736 responsive to suction (e.g., vacuum) force applied at the outlet of the conduit 736.

The suction force may be applied to the outlet of the conduit 736 by the vacuum source 774 either directly or indirectly. The suction force may be applied indirectly via the fluid storage container 772. For example, the outlet of the conduit 736 may be disposed within the fluid storage container 772 and an additional conduit 736 may extend from the fluid storage container 772 to the vacuum source 774. Accordingly, the vacuum source 774 may apply suction to the fluid collection assembly 700 via the fluid storage container 772. The suction force may be applied directly via the vacuum source 774. For example, the outlet of the conduit 736 may be disposed within the vacuum source 774. An additional conduit 736 may extend from the vacuum source 774 to a point outside of the fluid collection assembly 700, such as to the fluid storage container 772. In such examples, the vacuum source 774 may be disposed between the fluid collection assembly 700 and the fluid storage container 772.

The fluid collection assembly 700 may be similar or identical to any of the fluid collection devices disclosed herein in one or more aspects. For example, the fluid collection assembly 700 may include a fluid impermeable barrier at least partially defining a chamber of the fluid collection assembly 700. The fluid impermeable barrier also defines an opening extending therethrough. The opening may be positioned adjacent to or have a penis positioned therethrough. The fluid collection assembly 700 may include at least one porous material disposed within the fluid impermeable barrier. The conduit 736 includes an inlet and an outlet, the outlet being fluidly coupled to the fluid storage container and the inlet being positioned at or near a portion of the chamber selected to be at a gravimetrically low point of the fluid collection assembly 700 when worn.

The fluid storage container 772 is sized and shaped to retain a fluid therein. The fluid storage container 772 may include a bag (e.g., drainage bag), a bottle or cup (e.g., collection jar), or any other enclosed container for storing the bodily fluids. In some examples, the conduit 736 may 736 extend from the fluid collection assembly 700 and attach to the fluid storage container 772 at a first point therein. An additional conduit 736 may attach to the fluid storage container 772 at a second point thereon and may extend and attach to the vacuum source 774. Accordingly, a vacuum (e.g., suction) may be drawn through fluid collection assembly 700 via the fluid storage container 772. Fluid, such as urine, may be drained from the fluid collection assembly 700 using the vacuum source 774.

The vacuum source 774 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 774 may provide a vacuum or suction to remove fluid from the fluid collection assembly 700. In some examples, the vacuum source 774 may be powered by one or more of a power cord (e.g., connected to a power socket), one or more batteries, or even manual power (e.g., a hand operated vacuum pump). In some examples, the vacuum source 774 may be sized and shaped to fit outside of, on, or within the fluid collection assembly 700. For example, the vacuum source 774 may include one or more miniaturized pumps or one or more micro pumps. The vacuum sources 774 disclosed herein may include one or more of a switch, a button, a plug, a remote, or any other device suitable to activate the vacuum source 774.

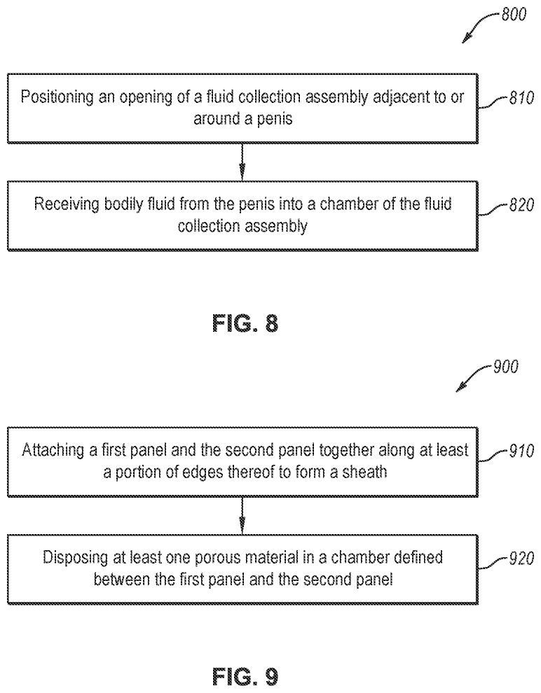

FIG. 8 is a flow diagram of a method 800 to collect fluid, according to an embodiment. The method 800 of collecting fluid may utilize use any of the fluid collection assemblies and/or fluid collection systems disclosed herein. The method 800 may include act 810, which recites “positioning an opening of a fluid collection assembly adjacent to or around a penis.” Act 810 may be followed by act 820, which recites “receiving bodily fluids from the penis into a chamber of the fluid collection assembly.”

Acts 810 and 820 of the method 800 are for illustrative purposes. For example, the acts 810 and 820 of the method 800 may be performed in different orders, split into multiple acts, modified, supplemented, or combined. In an example, one or more of the acts 810 or 820 of the method 800 may be omitted from the method 800.

Act 810 recites “positioning an opening of a fluid collection assembly adjacent to or around a penis.” The act 810 of positioning an opening of a fluid collection assembly may include utilizing any of the fluid collection assemblies or systems disclosed herein. In some examples, act 810 may include positioning the opening such that the porous material of the fluid collection assembly extends across the opening which allows the porous material to abut or be positioned proximate to a buried penis. In some examples, act 810 may include positioned a base of a fluid collection assembly over a buried penis such that the male urethra is positioned adjacent to an aperture of the base and an opening of the sheath. In such an example, act 810 may include positioning a sheath of the male fluid collection assembly around a non-buried penis, such that at least a portion of the penis is positioned through an opening of the sheath and in the chamber of the fluid collection assembly.

Act 820 recites, “receiving bodily fluids from the penis into a chamber of the fluid collection assembly.” In some examples, receiving bodily fluids from the penis into a chamber of the fluid collection assembly includes receiving the bodily fluids through the opening of the fluid collection assembly. Receiving bodily fluids from the penis into a chamber of the fluid collection assembly may include at least one of wicking, absorbing, or adsorbing the bodily fluids away from the opening using the porous material. In some examples, receiving bodily fluids from the penis into a chamber of the fluid collection assembly may include receiving the bodily fluids into the chamber of the sheath of the fluid collection assembly. Receiving bodily fluids from the penis into a chamber of the fluid collection assembly may include flowing the bodily fluids towards a portion of the chamber that is fluidly coupled to an inlet of a conduit in fluid communication a vacuum source. For instance, receiving bodily fluids from the penis into a chamber of the fluid collection assembly may include flowing the bodily fluids to a gravimetrically low point of the chamber, etc., such as via gravity, wicking, or suction force.

The method 800 may include applying suction with a vacuum source effective to suction the bodily fluids from the chamber via a conduit disposed or otherwise in fluid communication with the chamber. The conduit may also be fluidly coupled to the vacuum source may include using any of the vacuum sources disclosed herein. Applying suction with a vacuum source may include activating the vacuum source (e.g., suction device) in fluid communication with the inlet of the conduit in the fluid collection assembly. In some examples, activating the vacuum source in fluid communication with the inlet of the conduit in the fluid collection assembly may include supplying power to the vacuum source by one or more of flipping an on/off switch, pressing a button, plugging the vacuum source into a power outlet, putting batteries into the vacuum source, etc. In some examples, the vacuum source may include a hand operated vacuum pump and applying suction with a vacuum source may include manually operating the hand operated vacuum pump effective to suction the bodily fluids from the chamber via the conduit disposed therein that is fluidly coupled to the vacuum source.

In some examples, applying suction with a vacuum source effective to suction the bodily fluids from the chamber via a conduit disposed therein and fluidly coupled to the vacuum source may be effective to remove at least some bodily fluids from the chamber of the fluid collection assembly. In some examples, applying suction with a vacuum source effective to suction the bodily fluids from the chamber via a conduit disposed therein and fluidly coupled to the vacuum source may be effective to transfer at least some of the bodily fluids from the chamber to a fluid storage container (e.g., a bottle or bag).

In some examples, the vacuum source (e.g., suction device) may be disposed on or within the fluid collection assembly and applying suction with the vacuum source may include activating the vacuum source. In some examples, the vacuum source may be spaced from the fluid collection assembly and applying suction with the vacuum source may include activating the vacuum source.

In some examples, applying suction with a vacuum source effective to suction the bodily fluids from the chamber via a conduit disposed therein and fluidly coupled to the vacuum source may include detecting moisture in the chamber (e.g., via one or more moisture sensors) and responsive thereto, activating the vacuum source to provide suction in the chamber. The control of the vacuum source responsive to the signals indicating that moisture or a level thereof is present in the chamber may be automatic, such as via a controller (e.g., computer programmed to perform the operation), or may merely provide an indication that a level of moisture is present that may necessitate removal of bodily fluids from the chamber of the fluid collection assembly. In the latter case, a user may receive the indication (e.g., from the controller) and activate the vacuum pump manually.

In an example, the method 800 may include collecting the bodily fluids that are removed from the fluid collection assembly, such as into a fluid storage container that is spaced from the fluid collection assembly and fluidly coupled to the conduit. The fluid storage container may include any of the fluid storage containers disclosed herein.

FIG. 9 is a flow diagram of a method 900 to manufacture a fluid collection assembly, according to an embodiment. The method 900 may be used to manufacture at least some of the fluid collection assemblies and/or fluid collection systems disclosed herein. The method 900 may include act 910, which recites “attaching a first panel and the second panel together along at least a portion of edges thereof to form a sheath.” Act 910 may be followed by act 920, which recites “disposing at least one porous material in a chamber defined between the first panel and the second panel.”

Acts 910, 920 of the method 900 are for illustrative purposes. For example, the acts 910, 920 of the method 900 may be performed in different orders, split into multiple acts, modified, supplemented, or combined. In an example, one or more of the acts 910, 920 of the method 900 may be omitted from the method 900. Any of the acts 910 or 920 may include using any of the fluid collection devices or systems disclosed herein.

Act 910 recites “attaching at least a portion of a first panel and at least a portion of the second panel together along at least a portion of edges thereof to form a sheath.” In an example, when the first and second panels are distinct sheets, act 910 may include positioning the first panel and the second panel adjacent to each other (e.g., on top of each other). Positioning the first panel and the second panel adjacent to each other forms a chamber between the first panel and the second panel. After positioning the first and second panels, the first and second panels may be attached to each other, such as attached to each other along at least a portion of the outer edges thereof. For instance, all of the outer edges of the first and second panels may be attached together except for portions of the outer edges thereof that define an opening configured to receive a penis (if the opening is not completely defined by one of the first or second panels) and a fluid outlet. In an example, the first and second panels are integrally formed together. In such an example, act 910 may include attaching edges of the first and second panels that are not already attached. When the first and second panels are formed by a thin walled tube, act 910 may include attaching the portions of the first and second panels that form the proximal and distal end regions thereof together. When the first and second panels are formed by a single folded sheet, act 910 may include attaching the portions of the first and second panels that form the proximal and distal end regions thereof together and attaching the portions of the first and second panels that are opposite the fold.

The first and second panels may be attached together using any suitable technique. In an embodiment, the first and second panels are attached together by sewing the first and second panels together. In an embodiment, the first and second panels are attached together using a non-sewing technique, such as via heat sealing, RF welding, or US welding. Using a non-sewing technique to attach the first and second panels together may increase the rate of manufacture of the fluid collection assembly formed during the method 900. For example, heat sealing, RF welding, or US welding may be performed significantly faster than sewing the first and second panels together. Further, heat sealing, RF welding, or US welding may form better water proof seals compared to sewing.

Act 920 recites “disposing at least one porous material in a chamber defined between the first panel and the second panel.” In an embodiment, the porous material may be attached to one of the first or second panels (e.g., with an adhesive, heat sealing, RF welding, US welding, or any other suitable technique) before act 910. In such an embodiment, positioning the first and second panels adjacent to each other also disposes the porous material in the chamber. In an embodiment, the porous material may be positioned between the first and second panels when the first and second panels are positioned adjacent to each other. In other words, the first panel, the porous material, and the second panel may form a stack with the porous material positioned between the first and second panels. Act 920 may also attach the porous materials to the first and second panels as the first and second panels are attached together. As such, the method 900 does not require a separate act of attaching the porous material to the first and second panels thus making the method 900 more efficient and quicker. In an embodiment, the porous material is disposed in the chamber after attaching the first and second panels together. In such an embodiment, the porous material may be attached to one or more of the first or second panels with an adhesive, tape, or the like or the porous material may not be attached to the first and second panels. While not attaching the porous material to the first and second panels may make the method 900 more efficient and quicker, the porous material may move in the chamber, such as by bunching up near the outlet.

In an embodiment, the method 900 may include forming one or more of the first panel, the second panel, and the porous material. In an example, the method 900 may form the first and second panel, for example, by stamping the first and second panels (including any openings, cutouts, or perforation defined thereby). Stamping the first and second panels and attaching the first and second panels together, as discussed above, may be quicker and easier than forming a single sheet into a cylinder. In an example, the porous material may also be stamped from a sheet of the porous material when the porous material exhibits a sheet-like shape. In an example, the first and second panel may be formed using a blown film extrusion technique to form a thin walled tube. In an example, at least one of the first panel, the second panel, or the porous material may be formed by non-stamping techniques or non-blown film extrusion technique even though such techniques may be more complicated or time consuming Examples of non-stamping and non-blown film extrusion techniques include cutting with a blade, other extrusion techniques, molding (e.g., cast molding), or any other suitable technique.

In an embodiment, the method 900 may include forming the base. In an example, the base may be formed by stamping the base from a sheet (e.g., a sheet that includes a substrate, at least one adhesive layer, and at least one release liner). The base may be formed by stamping since, for instance, the base is formed from a thin film and the base may not include any protrusions, such as a ring-like protrusion defining the aperture. Alternatively, the base may be formed by injection molding, or any other suitable technique even though non-stamping techniques may be more time consuming.

In an embodiment, the method 900 may include permanently attaching the base to the sheath formed at least partially by acts 910 and 920. In an example, the method 900 includes permanently attaching the base to the sheath before providing the fluid collection assembly to an end user. In such an example, the method 900 may include permanently attaching the base to the sheath and, after attaching the base to the sheath, disposing the fluid collection assembly in a package. In an example, the method 900 does not include permanently attaching the base to the sheath before providing the fluid collection assembly to an end user. In such an example, the method 900 may include disposing the fluid collection assembly, with the sheath and the base not attached together, in a package. The end user may remove the fluid collection assembly from the package and may permanently attaching the base to the sheath after removing the fluid collection assembly from the package. For instance, the end user may permanently attach the sheath to the base before, during, or after attaching the base to the skin surround the penis.

It is noted that the embodiments disclosed above relate to fluid collection assemblies configured to collection bodily fluids from a male. However, it is noted that such fluid collection assemblies may also be used to collection bodily fluids from a female since the urethral opening of the female is, functionally, similar to a buried penis.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a sheath including:
at least one fluid impermeable barrier including a first panel and a second panel, the at least one fluid impermeable barrier defining a chamber at least partially defined by the first panel and the second panel, the at least one fluid impermeable barrier defining an outlet at a distal end of the sheath and an opening at a proximal end of the sheath, the proximal end of the sheath including proximal edges opposite the distal end that are attached together; and
at least one porous material disposed in the chamber, the at least one porous material including a sheet, the sheet configured to lie flat and extend across the opening when the sheath is resting on a flat surface and a penis is not disposed in the chamber, at least a portion of the at least one porous material is hydrophobic;
a base permanently secured to or configured to be permanently secured to the proximal end of the sheath, the base configured to be attached to skin surrounding a penis, the base defining an aperture that corresponds to the opening of the sheath.

2. The fluid collection assembly of claim 1, wherein at least a portion of the at least one fluid impermeable barrier is permeable to air.

3. (canceled)

4. The fluid collection assembly of claim 1, wherein the second panel is at least partially transparent.

5. The fluid collection assembly of claim 4, wherein the second panel is configured to be positioned adjacent to an individual using the fluid collection assembly during use.

6. (canceled)

7. The fluid collection assembly of claim 1, wherein the first panel and the second panel are formed from distinct sheets.

8. The fluid collection assembly of claim 1, wherein the first panel and the second panel are integrally formed.

9. (canceled)

10. The fluid collection assembly of claim 1, wherein, when a penis is inserted into the chamber through the opening, the penis is positioned between at least a portion of the second panel and the at least one porous material.

11. (canceled)

12. (canceled)

13. The fluid collection assembly of claim 1, wherein the at least one porous material includes a first layer, a second layer, and a plurality of fibers forming a layer between the first layer and the second layer.

14. The fluid collection assembly of claim 14, wherein the at least one porous material includes at least one of a hydrophobic foam or cotton.

15. (canceled)

16. (canceled)

17. The fluid collection assembly of claim 1, wherein the base includes:
a substrate defining a top surface and a bottom surface, wherein, during use, the top surface is configured to be closer to the sheath than the bottom surface and the bottom surface is configured to be closer to skin surrounding a penis than the top surface; and
a first adhesive layer attached at least a portion of the bottom surface, the first adhesive layer configured to attach the base to the skin surrounding the penis.

18. The fluid collection assembly of claim 17, wherein the first adhesive layer includes at least one of a silicone-based adhesive, an acrylic gel adhesive, or a hydrogel.

19. (canceled)

20. (canceled)

21. The fluid collection assembly of claim 1, wherein the base exhibits a generally triangular shape.

22. (canceled)

23. The fluid collection assembly of claim 1, wherein the sheath is free from at least one of one or more rings exhibiting a rigidity that is greater than the at least one fluid impermeable barrier and the at least one porous material, or a cap exhibiting a rigidity that is greater than the at least one fluid impermeable barrier and the at least one porous material.

24. (canceled)

25. (canceled)

26. (canceled)

27. A system, comprising:
the fluid collection assembly of claim 1:
a vacuum source configured to apply a suction force;
a fluid storage container; and
at least one conduit connected to the outlet and in fluid communication with the vacuum source and the fluid storage container.

28. A method of manufacturing a fluid collection assembly, the method comprising:
attaching a first panel and a second panel of a fluid impermeable barrier together along at least a portion of edges thereof to form a sheath, the first and second panels at least partially defining a chamber therebetween, wherein the fluid impermeable barrier defines an opening and an outlet, wherein the proximal end of the sheath includes proximal edges opposite the distal end that are attached together; and
disposing at least one porous material in the chamber, the at least one porous material including a sheet, the sheet configured to lie flat and extend across the opening when the sheath is resting on a flat surface and a penis is not disposed in the chamber, at least a portion of the at least one porous material is hydrophobic.

29. (canceled)

30. (canceled)

31. The method of claim 28, wherein the first panel and the second panel are formed from a thin walled tube.

32. The method of claim 28, wherein attaching the first panel and the second panel together includes at least one of heat sealing, RF welding, or ultrasonic welding the first panel to the second panel.

33. The method of claim 28, wherein disposing the at least one porous material in the chamber includes, before attaching the first panel and the second panel together, positioning the at least one porous material between the first panel and the second panel; and
wherein attaching the first panel and the second panel together includes attaching the at least one porous material to at least one of the first panel or the second panel.

34. The method of claim 28, further comprising permanently attaching a base to the sheath.

35. (canceled)

36. (canceled)

37. (canceled)

38. A method of using a system to collect bodily fluids from an individual, the method comprising:
attaching a base to skin surrounding a penis, the base permanently secured to or configured to be permanently secured to a sheath, the sheath comprising:
at least one fluid impermeable barrier including a first panel and a second panel, the at least one fluid impermeable barrier defining a chamber at least partially defined by the first panel and the second panel, the at least one fluid impermeable barrier defining a fluid outlet at a proximal end of the sheath and an opening at a distal end of the sheath, the proximal end of the sheath including proximal edges opposite the distal end that are attached together; and
at least one porous material disposed in the chamber, the at least one porous material including a sheet, the sheet configured to lie flat and extend across the opening when the sheath is resting on a flat surface and a penis is not disposed in the chamber, at least a portion of the at least one porous material is hydrophobic.

39. (canceled)

40. (canceled)

41. (canceled)
